(19) United States
(12) Patent Application Publication
(10) Pub. No.: US 2023/0081377 A1
(43) Pub. Date: Mar. 16, 2023

(54) METHODS FOR AMPLIFYING IMMUNOASSAY SIGNALS

(71) Applicant: Siemens Healthcare Diagnostics Inc., Tarrytown, NY (US)

(72) Inventors: Huey Lee, Millwood, NY (US); Michelle Son, Hopewell Junction, NY (US)

(73) Assignee: Siemens Healthcare Diagnostics Inc., Tarrytown, NY (US)

(21) Appl. No.: 17/904,518

(22) PCT Filed: Feb. 1, 2021
(86) PCT No.: PCT/US2021/070105

§ 371 Date: Aug. 18, 2022

Related U.S. Application Data
(60) Provisional application No. 62/985,069, filed on Mar. 4, 2020.

(51) Int. Cl. G01N 33/543; G01N 33/58

(52) U.S. Cl. CPC G01N 33/5432; G01N 33/582; G01N 33/586; G01N 2458/00

(57) ABSTRACT

Disclosed herein are methods for using modified liposomes or carrier proteins comprising (i) an acridinium ester (AE), and (ii) a first agent encapsulated by the liposomes and/or (iii) a second agent on the surface of the liposomes or the carrier proteins. Specifically, the disclosed methods provide methods of labeling a target of interest, assaying a biological sample for a target antigen, and detecting a target antigen in a biological sample. Further disclosed herein are methods for increasing the strength of a signal detected by an imaging modality.

### TECHNICAL FIELD

Disclosed herein are methods and kits for amplifying immunoassay labeling and detecting an analyte in a sample using preparation of liposomes encapsulating hydrophilic acridinium esters and proteins carrying acridinium esters.

### BACKGROUND

Immunoassay remains the method of choice in the clinical laboratory for analysis of many analytes, particularly complex heterogeneous molecules. A lack of or a low immunoassay signal and sensitivity can be a major obstacle for accurately diagnosing and prognosing a disease. There is a constant need in the art for improved immunoassay labeling methods that provide quick and reliable results which would benefit both patients and healthcare providers.

### SUMMARY

Disclosed herein are methods of detecting an analyte in a sample. The methods comprise (a) combining, in a medium, the sample with a conjugate reagent, a linker reagent, an amplifying reagent, and, optionally, a capture binding partner for the analyte; and (b) examining the medium for bound analyte, the bound analyte comprising the analyte bound to the conjugate reagent bound to the linker reagent bound to the amplifying reagent, wherein the conjugate reagent comprises a detection binding partner for the analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

Disclosed herein are kits comprising (a) a conjugate reagent; (b) a linker reagent; (c) an amplifying reagent; and optionally, a capture binding partner, wherein the conjugate reagent comprises a detection binding partner for a target analyte and a first small molecule, wherein the linker reagent comprises a binding partner for the small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

In some embodiments, the detection binding partner for the analyte comprises an antibody that specifically binds the analyte.

In some embodiments, the first small molecule and the second small molecule comprise biotin. In additional embodiments, the binding partner for the first small molecule and the second small molecule comprises streptavidin.

In some embodiments, the first small molecule and the second small molecule comprise fluorescein. In additional embodiments, the binding partner for the first small molecule and the second small molecule comprises anti-fluorescein antibody.

In some embodiments, the capture binding partner for the analyte further comprises a support. In further embodiments, the support is a non-magnetic particle, a magnetic particle, a plate, or a tube.

In some embodiments, the conjugate reagent further comprises a labeling agent.

In some embodiments, the linker reagent comprises a labeling agent.

In some embodiments, the labeling agent(s) comprises acridinium ester (AE).

In some embodiments, the diameter of the liposome is about 20 nm to about 1000 nm.

In some embodiments, the encapsulated AE has a concentration ranging from at least 1×10⁻⁸ mol/L to at least 1×10⁻⁶ mol/L.

In some embodiments, the liposome encapsulates about 1000 to about 100,000,000,000 hydrophilic AE molecules.

In some embodiments, the carrier protein comprises a bovine serum albumin (BSA).

In some embodiments, the carrier protein binds at least 1 to about 100 AE molecules.

In some embodiments, the disclosed methods further comprise a washing step prior to the step of examining the medium for bound analyte.

In other embodiments of the disclosed methods, the sample, the conjugate reagent, the linker reagent, the amplifying reagent, and optionally the capture binding partner are combined simultaneously or sequentially.

### BRIEF DESCRIPTION OF THE DRAWINGS

The summary, as well as the following detailed description, is further understood when read in conjunction with the appended drawings. For the purpose of illustrating the disclosed devices, systems, and methods, there are shown in the drawings exemplary embodiments of the devices, systems, and methods; however, the devices, systems, and methods are not limited to the specific embodiments disclosed. In the drawings:

FIG. 1 is a diagram illustrating an example of crosslinking based on linker protein streptavidin. Ag=antigen; Ab=antibody; AE=acridinium ester.

FIG. 2 is a series of diagrams illustrating how the crosslinking with linker protein streptavidin may be used in the presently disclosed compositions and methods.

FIG. 3 is a diagram illustrating an example of crosslinking based on linker protein anti-fluorescein antibody. Ag=antigen; Ab=antibody; AE=acridinium ester.

FIG. 4 is a series of diagrams illustrating how the crosslinking with anti-fluorescein antibody may be used in the presently disclosed compositions and methods.

FIG. 5 is a series of graphs depicting DLS's of biotinylated unilamellar liposomal vesicles (LUV)±avidin and fluoresceinated (FL) liposomal vesicles LUV±anti-fluorescein polyclonal antibody (anti-FL). Cross-linking of respective LUV's is observed. Anti-FL antibody can be monoclonal or polyclonal. F1 represents the first fraction collected from the purification. FTIC and FL are similar compounds.

FIG. 6 is an image and a table illustrating the disclosed assay. In experiment A (Exp A in table), AE trapped biotinylated vesicles can bind to DYNAL® beads M270 (Thermo Fisher Scientific)particles (streptavidin-coated magnetic latex particles). As shown in the picture, the detector is Berthold Autolumat Plus LB 953 (with a magnetic rack on top used for the manual assay). The bindings shown by the output, in relative light unit (RLU), are proportional to the amount of AE trapped biotinylated vessicles added. In experiment B (Exp B in table), decreasing M270 particles reduces the output signal.

FIG. 7 is a table demonstrating that the addition of linker protein streptavidin boosts signal output. In experiment A (Exp A in table), anti-FL paramagnetic particles (pmp) are used to capture AE trapped biotinylated and fluoresceinated vesicles. The signal is generated and amplified when linker protein streptavidin was added. In experiment B (Exp B in table), increasing the concentration of the linker protein increases signal amplification. Experiments were performed at room temperature (RT).

FIG. 8 is a graph depicting the competitive binding of fluorescein and fluoresceinated AE vesicles to the anti-FL pmp.

FIG. 9 is a graph depicting the amplification scheme demonstrated on Biacore® (an optical biosensor from General Electric Healthcare). Using a sensorchip immobilized with fluoresceinated BSA, a protein hapten (anti-FL Mab (2H1) conjugated to neutravidin) is first added to the chip, followed by couple injections of the biotinylated microbubble (stage 1 amplification). Additional neutravidin is introduced so that more biotinylated microbubble can bind (stage 2 amplification). Finally, stage 3 amplification is simply a repeat of stage 2 amplification.

FIG. 10 is a series of diagrams and a table illustrating the disclosed assay's set up on Siemens' automated system Centaur. TSH=thyroid stimulating hormone; TSH1,TSH5, & TSH10 (in the table below)=TSH standards of increasing concentrations; lite reagent (LR)=AE labeled streptavidin. Here, the particles immobilized with anti-TSH Pab (polyclonal antibody) can bind the antigen TSH, which then form sandwich with the biotinylated anti-TSH Mab (monoclonal antibody). Addition of AE labeled streptavidin produces the signals.

FIG. 11 is a series of diagrams and a table illustrating the introduction of linker protein and AE labeled amplifiers on Siemens' automated system Centaur. Linker protein=unlabeled streptavidin in ancillary well (AW); amplifier=AE's-BSA-biotin's; top figure=control; bottom figure=biotinylated anti-TSH Mab+the amplifier in the LR well. Linker protein streptavidin (without AE label in this case) is in the AW.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the singular forms “a,” “an,” and “the” include the plural referents unless context clearly indicates otherwise. Similarly, the word “or” is intended to include “and” unless the context clearly indicates otherwise.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term acridinium ester, as disclosed herein, refers to any acridinium ester which can be encapsulated within a liposome and which can generate a chemiluminescent signal.

The term “analyte” as used herein is a broad term and is used in its ordinary sense, including, without limitation, to refer to a detectable component or target of interest in a sample, such as a substance or chemical constituent in a biological liquid (for example, blood, interstitial liquid, cerebral spinal liquid, lymph liquid or urine). Analytes can include naturally occurring substances, artificial substances, metabolites, and/or reaction products. Examples of analytes include but are not limited to a ligand that is mono- or polyepitopic, antigenic, or haptenic or a nucleic acid such as DNA or RNA.

The term “solid support”, “support structure”, and “substrate” as used herein are used interchangeably and refer to a material or group of materials having a rigid or semi-rigid surface or surfaces. There is no limitation to the shape or size of the support structures. In many embodiments, the solid support(s) will take the form of beads (e.g., silica beads, magnetic beads, paramagnetic beads, and the like), resins, gels, microspheres, or other geometric configurations.

As used herein, a “functional group” refers to a chemical group within a molecule that is responsible for characteristic chemical reactions. Exemplary functional groups include, but are not limited to, those that contain an oxygen, a nitrogen, a phosphorus or a sulfur atom such primary amines, carboxyls, carbonyls, aldehydes, sulfhydryls, hydroxyl groups and esters. As used herein, a functional group is reactive with another group if the two groups can react to form a covalent bond.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

A “crosslinker” refers to a linker that joins two other molecules covalently.

As used herein, the term “linklification” refers to a signal amplification scheme of a given labeling agent.

The term “liposome” as used herein refers to an artificially formed vesicle or sac made up of a membrane comprising at least one lipid bilayer. The term is understood to exclude naturally occurring vesicles or other naturally occurring membranous substances isolated from cells or biological samples comprising cells. The terms “vesicle” and “liposome” can be synonymous as used herein in reference to the artificially formed sacs comprising a membrane of at least one lipid bilayer. For example, an artificially formed large unilamellar liposomal vesicle, or “LUV,” is termed a vesicle, but is also referred to as a liposome for purposes of this patent application.

Poly(ethylene glycol), commonly known as PEG, refers to an oligomer of ethylene oxide forming a linear chain. PEG molecules can be either linear or can be branched, wherein each molecule has at least two and generally three or more PEG branches or arms emanating from a central core group.

The term “antibody” refers to an immunoglobulin molecule which is able to specifically bind to a specific epitope on an antigen. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), camelid antibodies and humanized antibodies. As contemplated herein, an antibody conjugated to a quantum dot and support structure may specifically or non-specifically recognize and/or bind to an analyte, such that the analyte can be analyzed qualitatively and quantitatively.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

By “disease” is meant any condition or disorder that damages or interferes with the normal function of a cell, tissue, or organ.

“Detect” refers to identifying the presence, absence or amount of a target (e.g. an analyte to be detected.

An “individual”, “patient” or “subject”, as these terms are used interchangeably herein, includes a member of any animal species including, but are not limited to, birds, humans and other primates, and other mammals including commercially relevant mammals such as cattle, pigs, horses, sheep, cats, and dogs. Preferably, the subject is a human.

As used herein, the terms “treatment” and “treating” refer to an approach for obtaining beneficial or desired results including, but not limited to, therapeutic benefit and/or a prophylactic benefit. For example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

By therapeutic benefit is meant eradication or amelioration of the underlying disorder being treated or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Various terms relating to aspects of the description are used throughout the specification and claims. Such terms are to be given their ordinary meaning in the art unless otherwise indicated. Other specifically defined terms are to be construed in a manner consistent with the definitions provided herein.

### DETAILED DESCRIPTION

Provided herein are methods for amplifying immunoassay labeling and detecting an analyte in a sample using preparation of liposomes encapsulating hydrophilic acridinium esters (AEs) or protein carriers comprising AEs for the purpose of amplifying a signal.

The disclosed methods for detecting an analyte in a sample comprise (a) combining, in a medium, the sample with a conjugate reagent, a linker reagent, an amplifying reagent, and, optionally, a capture binding partner for the analyte; and (b) examining the medium for bound analyte, the bound analyte comprising the analyte bound to the conjugate reagent bound to the linker reagent bound to the amplifying reagent, wherein the conjugate reagent comprises a detection binding partner for the analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

In some embodiments, the detection binding partner for the analyte comprises an antibody that specifically binds the analyte. The antibody can be a monoclonal antibody, antibody fragment, a bispecific or a multispecific antibody, a dimeric, a tetrameric or a multimeric antibody, or single chain antibody capable of specifically binding to the analyte.

In some embodiments, the analyte can be an antigen from a biological sample. In some embodiments, the biological sample can be, but is not limited to, whole blood, serum, plasma, urine, saliva, semen, or cerebrospinal fluid.

In some embodiments, the disclosed methods are useful of various assays. The assays comprise a biochemical assay such as an immunoassay, a clinical chemistry assay or other medical or diagnostic test. In some embodiments, the assays can comprise a sandwich assay or an in-situ hybridization assay.

In some embodiments, the disclosed methods comprise a reaction mixture for a biochemical assay. The mixture can include one or more reagents or buffers for the assay and the biological sample.

In some embodiments, the amplifying reagent which comprises a liposome or a carrier protein, is added in suspension form to the biological sample, the reagent, or the reaction mixture for a biochemical assay. In some embodiments, the amplifying reagent can be reconstituted from “dry form” in the biological sample, the reagent, or the reaction mixture or in one or more components that contribute to the reaction mixture for the biochemical assay.

In some embodiments, the first small molecule and the second small molecule comprise a biotin, an avidin or an avidin derivative (e.g., neutravidin), In other embodiments, the binding partner for the first small molecule and the second small molecule further comprises streptavidin.

In some embodiments, the first small molecule and the second small molecule comprise fluorescein. In other embodiments, the binding partner for the first small molecule and the second small molecule further comprises anti-fluorescein antibody.

In some embodiments, the first small molecule and the second small molecule are conjugated with a polypeptide, an antibody or antigen-binding fragment thereof, an aptamer, an affibody, an affimer, a carbohydrate, a polyethylene glycol (PEG), or a PEGylated polypeptide. In some embodiments, the PEGylated polypeptide comprises PEGylated antibody or PEGylated biotin.

In some embodiments, the disclosed carrier proteins comprise small or large proteins (MW>100 kD), or polymers which can conjugate to an analyte. Suitable carrier proteins comprise, but are not limited to, chitin, chitosan, gelatin, albumin, bovine serum albumin (BSA), ferritin, α1-macroglobulin and thyroglobulin. Carrier proteins can be synthetic polymers such as polyvinyl alcohols, polyacrylates, polysulphonates, polyamides, polyesters and polyethers.

In some embodiments, the carrier protein comprises a serum albumin bovine (BSA).

In some embodiments, the labeling agent agent(s) comprises acridinium esters (AEs). AEs are stable compounds that provide superior immunoassay performance in the form of increased sensitivity when compared with radioisotopes. The use of AEs can be advantageous for a variety of applications such as labelling ligands or analytes (such as antigens); labelling the specific binding partners of ligands or analytes (such as the corresponding antibodies); or labelling nucleic acids and molecules comprising nucleic acids.

In some embodiments, the carrier protein binds at least 1 to at least about 10 AE molecules, at least 1 to at least about 20 AE molecules, at least 1 to at least about 30 AE molecules, at least 1 to at least about 40 AE molecules, at least 1 to at least about 50 AE molecules, at least 1 to at least about 60 AE molecules, at least 1 to at least about 70 AE molecules, at least 1 to at least about 80 AE molecules, at least 1 to at least about 90 AE at least 1 to at least about 100 AE molecules, at least 1 to at least about 200 AE molecules, at least 1 to at least about 300 AE molecules, at least 1 to at least about 400 AE molecules, at least 1 to at least about 500 AE molecules, at least 1 to at least about 600 AE molecules, at least 1 to at least about 700 AE molecules, at least 1 to at least about 800 AE molecules, at least 1 to at least about 900 AE molecules, at least 1 to at least about 1000 AE molecules.

In some embodiments, the carrier protein binds at least 1 to about 100 AE molecules.

In some embodiments, the labeling agent encapsulated by the liposome is a hydrophilic acridinium ester (AE). The hydrophilic nature of the AEs renders them suitable for encapsulation within liposomes without leakage through the liposome wall. Detailed description of hydrophilic AEs can be found in the art such as in U.S. Pat. No. 5,656,426 A, the disclosure of which is hereby incorporated by reference in its entirety.

In some embodiments, the concentration of hydrophilic AEs encapsulated by the liposomes is at least 1·10⁻¹⁰ mol/L to at least 1·10⁻⁹ mol/L, at least 1·10⁻⁹ mol/L to at least 1·10⁻⁸ mol/L, at least 1·10⁻⁸ mol/L to at least 1·10⁻⁷ mol/L, at least 1·10⁻⁷ mol/L to at least 1·10⁻⁶ mol/L, at least 1·10⁻⁶ mol/L to at least 1·10⁻⁵ mol/L, at least 1·10⁻⁵ mol/L to at least 1·10⁻⁴ mol/L, at least 1·10⁻⁴ mol/L to at least 1·10⁻³ mol/L, at least 1·10⁻³ mol/L to at least 1·10⁻² mol/L, and at least 1·10⁻² mol/L to at least 1·10⁻¹ mol/L. In other embodiments, the hydrophilic AEs have a concentration ranging from at least 1·10⁻⁸ mol/L to at least 1·10⁻⁶ mol/L.

In some embodiments, the liposomes can encapsulate at least 10 to at least 100 hydrophilic AE molecules, at least 100 to at least 1,000 hydrophilic AE molecules, at least 1,000 to at least 10,000 hydrophilic AE molecules, at least 10,000 to at least 100,000 hydrophilic AE molecules, at least 100,000 to at least 1,000,000 hydrophilic AE molecules, at least 1,000,000 to at least 10,000,000 hydrophilic AE molecules, at least 10,000,000 to at least 100,000,000 hydrophilic AE molecules, at least 100,000,000 to at least 1,000,000,000 hydrophilic AE molecules, at least 1,000,000,000 to at least 10,000,000,000 hydrophilic AE molecules, at least 10,000,000,000 to at least 100,000,000,000 hydrophilic AE molecules, and at least 100,000,000,000 to at least 1,000,000,000,000 hydrophilic AE molecules. In other embodiments, the modified liposomes comprise at least about 1000 to at least about 100,000,000,000 hydrophilic AE molecules.

In further embodiments, the liposomes can be of various sizes. In some embodiments, the diameter of the liposome is about 20 nm to about 1000 nm. In some embodiments, the diameter of the liposome is about 20 nm to about 30 nm; about 30 nm to about 40 nm; about 40 nm to about 50 nm; about 50 nm to about 60 nm; about 60 nm to about 70 nm; about 70 nm to about 80 nm; about 80 nm to about 90 nm; about 90 nm to about 100 nm; about 100 nm to about 110 nm; about 110 nm to about 120 nm; about 120 nm to about 130 nm; about 130 nm to about 140 nm; about 140 nm to about 150 nm; about 150 nm to about 160 nm; about 160 nm to about 170 nm; about 170 nm to about 180 nm; about 180 nm to about 190 nm; about 190 nm to about 200 nm; about 200 nm to about 250 nm; about 250 nm to about 300 nm; about 350 nm to about 400 nm; about 400 nm to about 450 nm; about 450 nm to about 500 nm; about 500 nm to about 550 nm; about 550 nm to about 600 nm; about 600 nm to about 650 nm; about 650 nm to about 700 nm; about 700 nm to about 750 nm; about 750 nm to about 800 nm; about 800 nm to about 850 nm; about 850 nm to about 900 nm; about 900 nm to about 950 nm; and about 950 nm to about 1000 nm. In other embodiments, the diameter of the liposome is about 10 nm to about 500 nm. In yet other embodiments, the diameter of the liposome is about 30 nm to about 100 nm.

In some embodiments, the liposomes useful for the disclosed methods include multilamellar liposomal vesicles (MLVs), small unilamellar liposomal vesicles (SUVs), large unilamellar liposomal vesicles ULUVs), and giant unilamellar liposomal vesicles (GUVs). In some embodiments, the lipid bilayer can comprise sphingolipids, glycerophospholipids, sterols, and sterol derivatives. Sphingolipids to be used can include sphingomyelin and ceramides containing saturated, monounsaturated, and/or polyunsaturated acyl chains of different lengths. Phospholipids with various headgroup structures can be used, including phosphatidic acid (PA), phosphatidylcholine (PC), phosphatidylethanolamine (PE), phosphatidylglycerol (PG), phosphatidylinositol (PI), cardiolipin, phosphatidylserine (PS) containing saturated, monounsaturated, and/or polyunsaturated acyl chains of different lengths. Sterols and sterol derivatives to be used can include cholesterol, brassicasterol, allocholesterol, cholesterol methyl ether, campestanol, campesterol, cholesteryl acetate, coprostanol, desmosterol, dehydrodesmosterol, dihydrocholesterol, dihydrolanosterol, epicholesterol, lathosterol, lanosterol, sitostanol, sitosterol, stigmasterol, zymostenol, and zymosterol.

The liposomes useful for the disclosed methods can comprise modified phospholipids. For example, sphingolipids and glycerophospholipids can be modified with small molecules, polyethylene glycol (PEG), fluorescent molecules, fluorescent PEG, and/or bromine. Sphingolipids and glycerophospholipids, sterols, sterol derivatives, and modified versions of lipids are readily available commercially from various sources, such as Sigma-Aldrich (St. Louis, Mo.); Invitrogen (Carlsbad, Calif.); Avanti Polar Lipids (Alabaster, Ala.); Fisher Scientific (Pittsburgh, Pa.); Steraloids (Newport, R.I.).

In some embodiments, the liposomes are ruptured, and the amount of signal generated by the encapsulated hydrophilic AE is measured.

In some embodiments, a peptide and/or a nucleic acid are detected using the disclosed modified liposomes. For instance, a DNA or RNA probe is tagged with a ligand such as a hapten or a biotinylated modified nucleotide. The DNA or RNA probe is allowed to hybridize with complementary DNA or RNA and immobilized on a solid support. The immobilized probe is then reacted with the modified liposomes comprising a receptor for the ligand, such as an antibody or if the probe is biotinylated, avidin. The liposomes are ruptured, and the amount of signal generated by the encapsulated acridinium ester is measured.

In some embodiments, the capture binding partner used in the disclosed methods for detecting an analyte in a sample further comprises a support. In further embodiments, the support is a non-magnetic particle, a magnetic particle, a plate, or a tube.

In some embodiments the analyte is captured by means known in the art. These means comprise immunoassay devices and methods which may utilize labeled molecules in various sandwich, competition, or other assay formats. Such assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. These methods comprise for instance biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (e.g. Siemens' platforms like ADVIA Centaur® XPT, ADVIA Centaur® XP, ADVIA Centaur® CP, IMMULITE® 1000, IMMULITE® 2000 XPi and Atellica®; or General Electric Healthcare's platforms like Biacore®), enzymatic Cobalt Binding Assay (CBA), and latex agglutination assays.

Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a gene in the test sample, the sequence that is present in the nucleic acid probe is also present in the mRNA of the subject. More than one nucleic acid probe can also be used.

In some embodiments, the disclosed methods further comprise a washing step prior to the step of examining the medium for bound analyte.

In other embodiments of the disclosed methods, the sample, the conjugate reagent, the linker reagent, the amplifying reagent, and optionally the capture binding partner are combined simultaneously or sequentially.

### Kits

In certain aspects of the disclosed methods, kits are provided. The disclosed kits comprise (a) a conjugate reagent; (b) a linker reagent; (c) an amplifying reagent; and optionally, a capture binding partner, wherein the conjugate reagent comprises a detection binding partner for a target analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

The disclosed kits are useful for detecting the presence of an analyte in sample. In some embodiments, the analyte will comprise an antigen, an antibody, a peptide, or polypeptide of interest.

In some embodiments, the kits comprise a panel of probe sets. Probe sets comprise a large or small number of probes that detect the analytes (e.g. peptides) of interest. Probe sets may also comprise a large or small number of probes that detect peptides that are not informative about the analyte of interest. Such probes are useful as controls and for normalization (e.g., spiked-in markers).

Probe sets may be a dry mixture or a mixture in solution. In some embodiments, probe sets can be affixed to a solid substrate to form an array of probes. The probes may be antibodies, or nucleic acids (e.g., DNA, RNA, chemically modified forms of DNA and RNA), LNAs (Locked nucleic acids), or PNAs (Peptide nucleic acids), or any other polymeric compound capable of specifically interacting with the analytes of interest.

It is contemplated that kits may be designed for isolating and/or detecting analytes in essentially any sample (e.g., urine, blood, etc.), and a wide variety of reagents and methods are, in view of this specification, known in the art.

The following examples further illustrate aspects of the present invention. However, they are in no way a limitation of the teachings or disclosure of the present disclosure as set forth herein.

### Illustrative Embodiments

Provided here are illustrative embodiments of the disclosed technology. These embodiments are illustrative only and do not limit the scope of the present disclosure or of the claims attached hereto.

Embodiment 1. A method of detecting an analyte in a sample, the method comprising: (a) combining, in a medium, the sample with a conjugate reagent, a linker reagent, an amplifying reagent, and, optionally, a capture binding partner for the analyte; and (b) examining the medium for bound analyte, the bound analyte comprising the analyte bound to the conjugate reagent bound to the linker reagent bound to the amplifying reagent, wherein the conjugate reagent comprises a detection binding partner for the analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

Embodiment 2. A kit comprising (a) a conjugate reagent; (b) a linker reagent; (c) an amplifying reagent; and optionally, a capture binding partner, wherein the conjugate reagent comprises a detection binding partner for a target analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

Embodiment 3. The method of embodiment 1 or the kit of embodiment 2, wherein the detection binding partner for the analyte comprises an antibody that specifically binds the analyte.

Embodiment 4. The method or kit of any preceding embodiment, wherein the first small molecule and the second small molecule comprise biotin.

Embodiment 5. The method or kit according to embodiment 4 wherein the binding partner for the small molecule comprises streptavidin.

Embodiment 6. The method or kit of any one of embodiments 1 to 3, wherein the first small molecule and the second small molecule comprise fluorescein.

Embodiment 7. The method or kit of claim 6 wherein the binding partner for the small molecule comprises anti-fluorescein antibody.

Embodiment 8. The method or kit of any preceding embodiment, wherein the capture binding partner for the analyte further comprises a support.

Embodiment 9. The method or kit of embodiment 8 wherein the support is a non-magnetic particle, a magnetic particle, a plate, or a tube.

Embodiment 10. The method of any one of embodiments 1, 3, 4, 5, 6, 7, 8, and 9, further comprising a washing step prior to the step of examining the medium for bound analyte.

Embodiment 11. The method or kit of any preceding embodiment, wherein the conjugate reagent further comprises a labeling agent.

Embodiment 12. The method or kit of any preceding embodiment, wherein the linker reagent comprises a labeling agent.

Embodiment 13. The method or kit of any preceding embodiment, wherein the carrier protein comprises a bovine serum albumin (BSA).

Embodiment 14. The method or kit of any preceding embodiment, wherein the labeling agent(s) comprises acridinium ester (AE).

Embodiment 15. The method of any one of embodiments 1, 3, 4, 5, 6, 7, 8, 9, 10, 11, 12, and 13, wherein the sample, the conjugate reagent, the linker reagent, the amplifying reagent, and optionally the capture binding partner are combined simultaneously or sequentially.

Embodiment 16. The method or kit of any preceding embodiment, wherein the diameter of the liposome is about 20 nm to about 1000 nm.

Embodiment 17. The method or kit of embodiment 14, wherein the encapsulated AE has a concentration ranging from at least 1×10⁻⁸ mol/L to at least 1×10⁻⁶ mol/L.

Embodiment 18. The method or kit of embodiment 14, wherein the liposome encapsulates about 1000 to about 100,000,000,000 hydrophilic AE molecules.

Embodiment 19. The method or kit of embodiment 14, wherein the carrier protein binds at least 1 to about 100 AE molecules.

### EXAMPLES

The following examples are provided to further describe some of the embodiments disclosed herein. The examples are intended to illustrate, not to limit, the disclosed embodiments.

### Materials and Methods

### Reagents

1,2-dipalmitoyl-sn-glycero-3-phosphocholine (DPPC); 1-palmitoyl-2-oleoyl-phosphatidylcholine (POPC); 1,2-dioleoyl-sn-glycero-3-phosphocholine (DOPC); 1-palmitoyl-2-oleoyl-sn-glycero-3-phospho-L-serine (POPS); Porcine brain sphingomyelin (SM); cholesterol (CHOL); 1,2-distearoyl-sn-glycero-3-phosphoethanolamine-N-[biotinyl(polyethylene glycol)-2000] (PEG 2000 Biotin-DSPE); 1,2-dipalmitoyl-sn-glycero-3-phosphoethanolamine-N-(biotinyl) (Biotin-DPPE); 1,2-dipalmitoyl-sn-glycero-3-phosphoethanolamine-N-(Rhodamine-DPPE); N-(Fluorescein-5-Thiocarbamoyl)-1,2-Dihexadecanoyl-sn-Glycero-3-Phosphoethanolamine (Fluorescein-DHPE); N-sulfo-propyl-dimethyl acridinium ester-N-hydroxysuccinimide (NSP-DMAE) and Trimethylsilyl propionic DMAE (TSP-DMAE).

Lipids were stored at −20° C. Various polycarbonate membrane filters were used with pore diameter 30, 50, 100, 200, and 400 nanometer.

### Preparation and Purification of Liposomes Encapsulating Acridinium Esters

To prepare acridinium ester-encapsulating 4 mM large unilamellar liposomal vesicles (LUVs), lipids (SM or DPPC or POPC or DOPC or SM/POPC 1/1 or DPPC/POPC 1/2 or POPC/POPS 3/1) were mixed and dried under nitrogen followed by high vacuum for at least 2 hours. The amount of cholesterol used in the liposome was varied between 0 and 50 mol % depending on the specific experiment. All the lipid mixtures contained 0.025 mol % of Rhodamine PE to track the final concentration of liposomes. The dried lipid films were dispersed in NSP-DMAE or TSP-DMAE containing phosphate-buffered saline (PBS, 137 mM NaCl, pH 7.4) at 70° C. and then cooled down to room temperature before use. The concentration of NSP-DMAE and TSP-DMAE was varied between 0 and 15 mg/mL. The lipid mixture was subjected to 10 cycles of freezing/thawing and then extruded through polycarbonate filters with certain pore diameter (e.g., 30 nm, 50 nm, 100 nm, 200 nm, and 400 nm) to obtain uniform liposome size. NAP-5 (Sephadex G-25) column was used to remove untrapped NSP-DMAE or TSP-DMAE. Dynamic light scattering (DLS) measurements were conducted both before and after NAP-5 column purification. The particle-size distribution of the liposomes obtained showed that the mean diameter of the liposomes was still maintained after the purification step.

Encapsulated AE liposomes were prepared in the presence of various functional groups on the surface of the liposomes. Biotin-DPPE, PEG 2000 Biotin-DSPE or Fluorescein-DHPE was added into the lipid mixtures, i.e. SM or DPPC or POPC or DOPC or SM/POPC 1/1 or DPPC/POPC 1/2 or POPC/POPS 3/1 with or without cholesterol as described above, before the lipids were dried under the nitrogen. The amount of cholesterol was varied between 0 and 50 mol %. The amount of Biotin-DPPE, PEG 2000 Biotin-DSPE or Fluorescein-DHPE used in the liposomes was varied between 0 and 20 mol %. The permeability and hydrophilicity of the surface of liposomes were particularly enhanced by the addition of polyethyleneglycol (PEG).

### Example 1: Characteristics of the Linklification System

Provided herein are methods and kits of detecting an analyte in a sample using a linklification system.

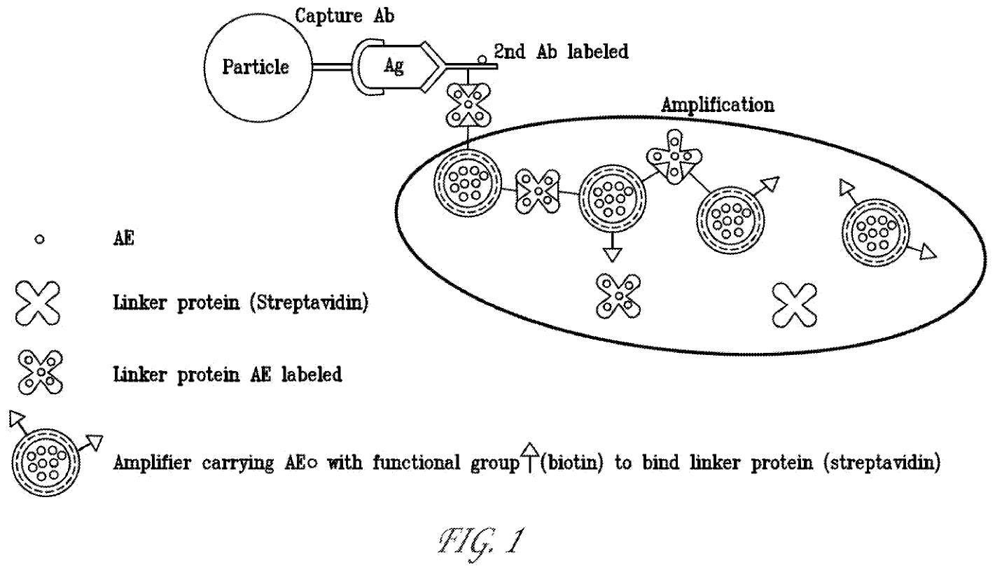

As shown in FIG. 1, streptavidin can be used as linker protein. Streptavidin is a tetrameric binding protein capable of binding four biotins and may be labeled with signal generating molecule AE as well. Specific assay antibody is biotinylated and may also be labeled with signal generating molecule AE (“2^{nd} Ab labeled”). Amplifier in this case can be biotinylated encapsulated AE liposomes (“Amplifier carrying AE”) or a hapten like AE(n)-BSA-biotin(n). A typical biotinylated liposome (100 nm i.d.) can easily carry more than 1000 AE molecules whereas the smaller hapten AE(n)-BSA-biotin(n) can have as many as 20-30 AE molecules per BSA. The number of biotins in both cases can be much less but is preferably at least two to enable crossing-linking with the linker protein. Streptavidin links the specific assay antibody to the amplifier and further links the amplifier to more amplifiers in a chain-like reaction in order to amplify signals on a system. The diagram of FIG. 1 shows the use of two binding sites on streptavidin with two free sites capable of binding two more amplifiers.

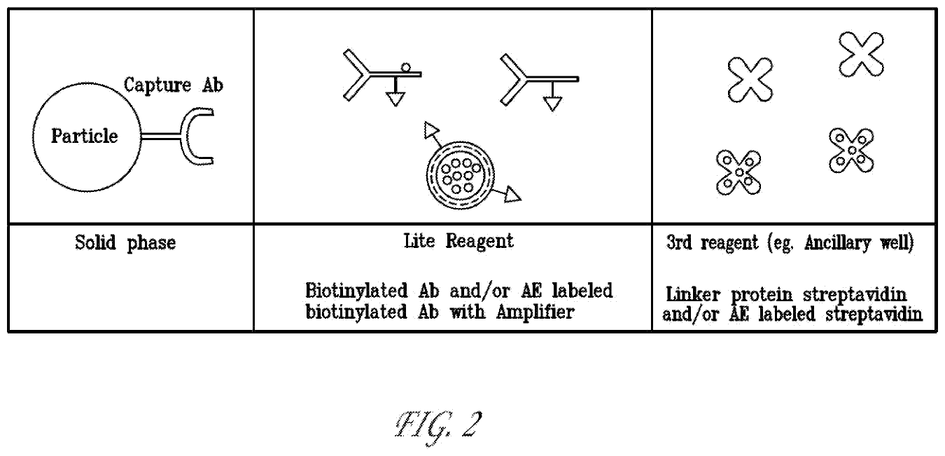

As shown in FIG. 2, in some embodiments, the assay system contains a solid phase compartment that contains the capture binding partner for the analyte with a solid support (the solid phase reagent or “SPR”). The signal generating lite reagent (LR) compartment of the assay system contains the LR antibody or antibodies that are also biotinylated. The LR antibody or antibodies may or may not be AE-labeled but must be biotinylated. Some LR antibodies may benefit from not being labeled with AE, which in general is more hydrophobic than molecules like biotin or fluorescein. The LR compartment can also contain the biotinylated amplifier carrying significantly more AE molecules (FIG. 2). The assay system further includes a third reagent compartment, for example, an ancillary well (“AW”), to separately contain the linker protein and/or AE-labeled linker protein, which will be introduced in the assay to mix with the lite reagent. The optimal molar ratios of all components involved should be determined experimentally. An example might be 1:1:1 (LR Ab:linker protein:amplifier).

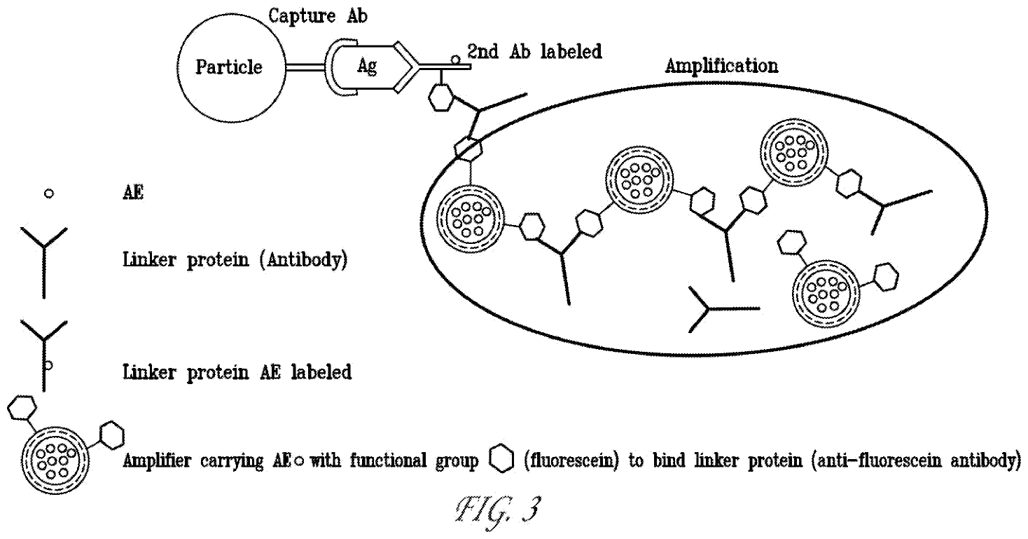

As shown in FIG. 3, anti-fluorescein antibody can be used as linker protein. Anti-fluorescein antibody (monoclonal or polyclonal) is a binary binding protein capable of binding two fluorescein molecules and may be labeled with signal generating molecule AE as well. Specific assay antibody is fluoresceinated and may also be labeled with signal generating molecule AE (“2^{nd} Ab labeled”). Amplifier in this case can be fluoresceinated encapsulated AE liposomes (“Amplifier carrying AE”) or a hapten like AE(n)-BSA-fluorescein(n). Anti-fluorescein antibody links the specific assay antibody to the amplifier and further links the amplifier to more amplifiers in a chain-like reaction in order to amplify signals on the system.

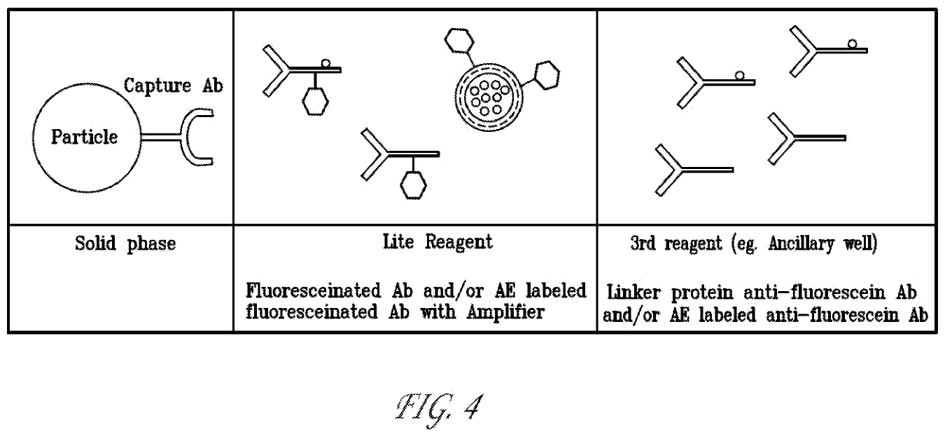

As shown in FIG. 4, the signal generating lite reagent (LR) compartment of the assay system contains the LR antibody or antibodies that are also fluoresceinated. The LR antibody or antibodies may or may not be AE-labeled but must be fluoresceinated. Some specific LR antibodies may benefit from not being labeled with AE, which in general is more hydrophobic than molecules like biotin or fluorescein. As shown, the LR compartment now also contains the fluoresceinated amplifier carrying significantly more AE molecules. The assay must add a new reagent compartment to contain the linker protein and/or AE-labeled linker protein separately, which will be introduced in the assay to mix with the lite reagent. The molar ratios of all components involved should be experimentally determined. An example might be 1:1:1 (LR Ab:linker protein:amplifier).

### Example 2: A Linklification System Based on Linker Proteins and Amplifiers Allows Amplification of Immunoassay Signals

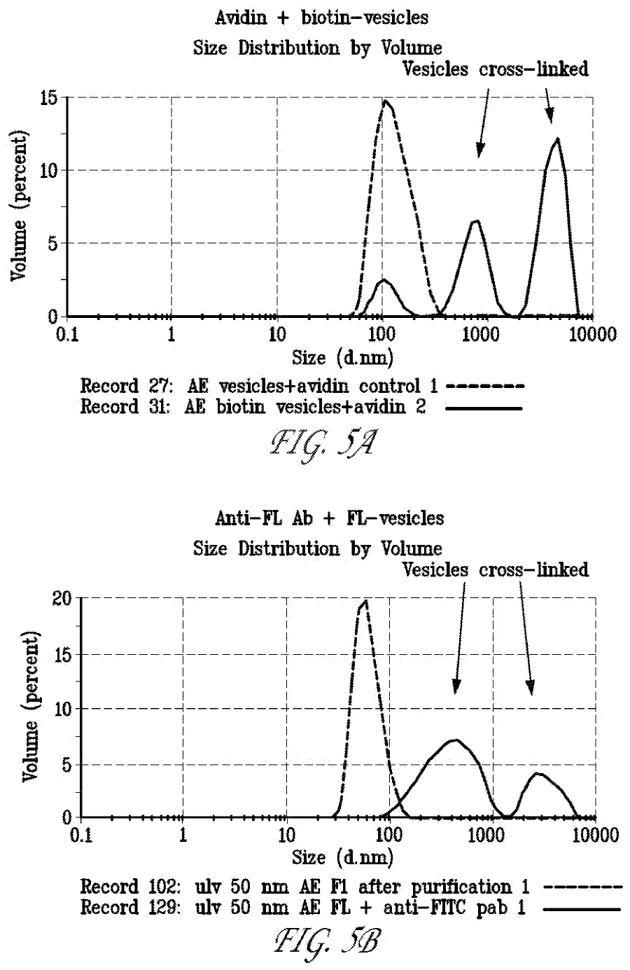

Liposomes of various sizes (20-1000 nm) can be useful for the disclosed methods and kits. The AE-encapsulating liposomes can also include additional modifications. These modifications include, but are not limited to, the addition of various functional groups such as biotin, fluorescein, and/or proteins on the liposomal surface (FIG. 5).

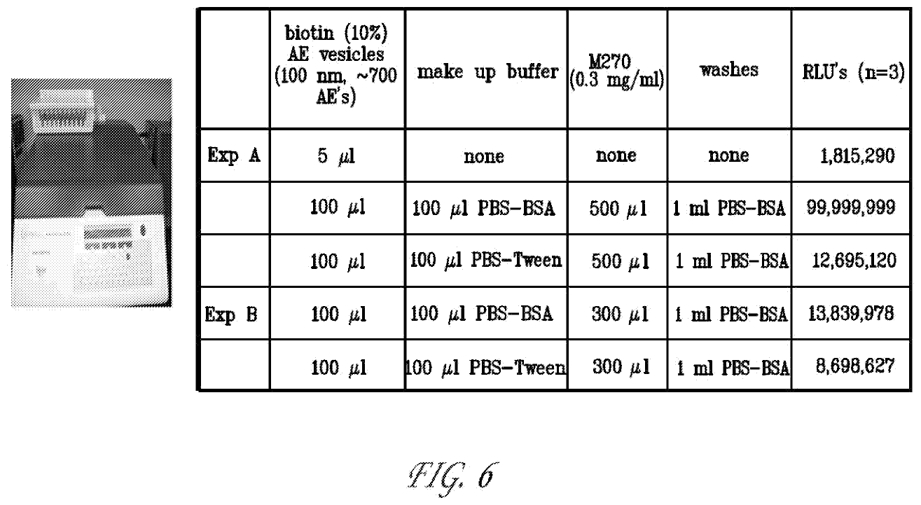

Encapsulated AE biotinylated liposomes (“biotin AE vesicles”) can bind to streptavidin-coated magnetic latex particles (Dynal's M270 particles, FIG. 6). The bindings shown by the output RLU are proportional to the amount of Encapsulated AE biotinylated liposomes added (Exp. A in FIG. 6). Decreasing the M270 particles reduces the output signal (Exp. B in FIG. 6).

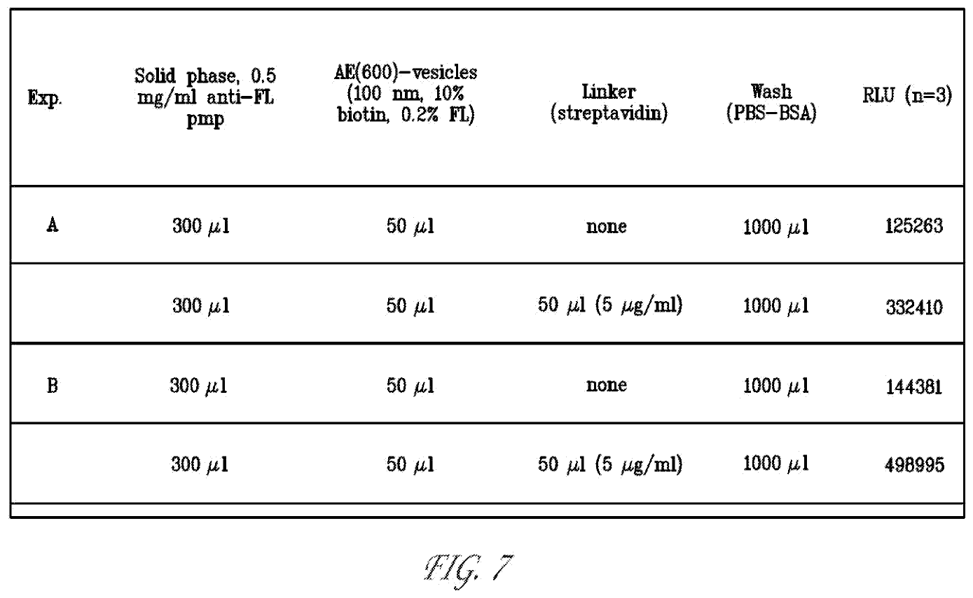

As shown by FIG. 7, the addition of linker protein streptavidin boosts the signal output. In experiment A, anti-FL paramagnetic particles (pmp) are used to capture encapsulated AE biotinylated and fluoresceinated liposomes. The signal is generated and amplified when linker protein streptavidin was added. In experiment B (Exp B in table), increasing the concentration of the linker protein increases signal amplification.

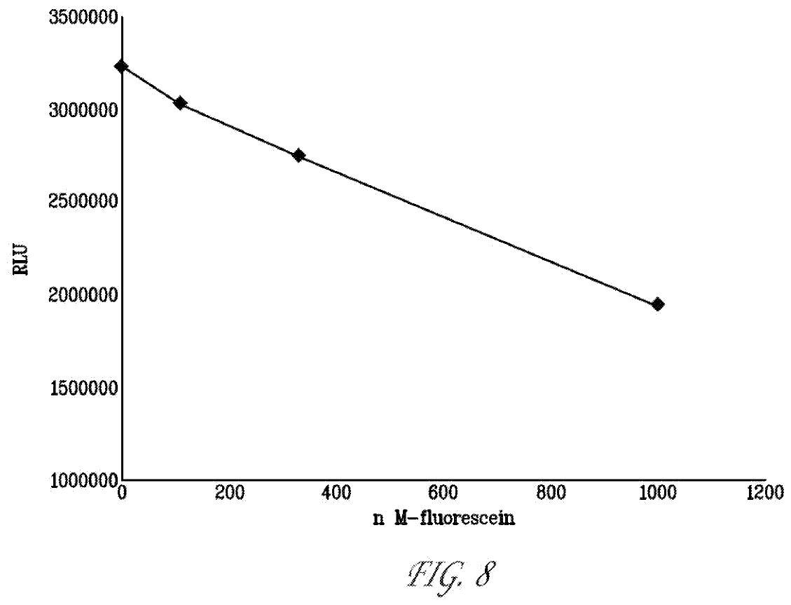

Furthermore, a competitive binding exists between fluorescein and fluoresceinated AE vesicles to the anti-FL paramagnetic particles (pmp), demonstrating that the fluoresceinated encapsulated AE liposomes are functional (FIG. 8).

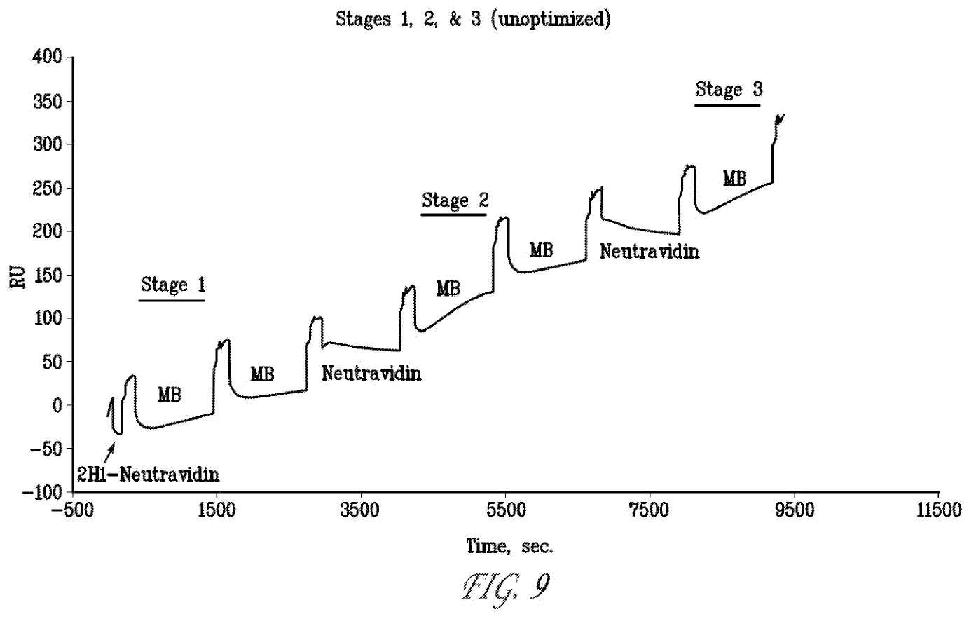

The disclosed linklification system (i.e. amplification system) is practical and does not seem to have specific signal amplification limitations (See FIG. 9). Using a sensorchip immobilized with fluoresceinated BSA, a protein hapten (anti-FL Mab (2H1) conjugated to neutravidin) is first added to the chip, followed by couple injections of the biotinylated microbubble (stage 1 amplification). Additional neutravidin is introduced so that more biotinylated microbubble can bind (stage 2 amplification). Finally, a stage 3 amplification is performed which is a simple repeat of stage 2 amplification (FIG. 9).

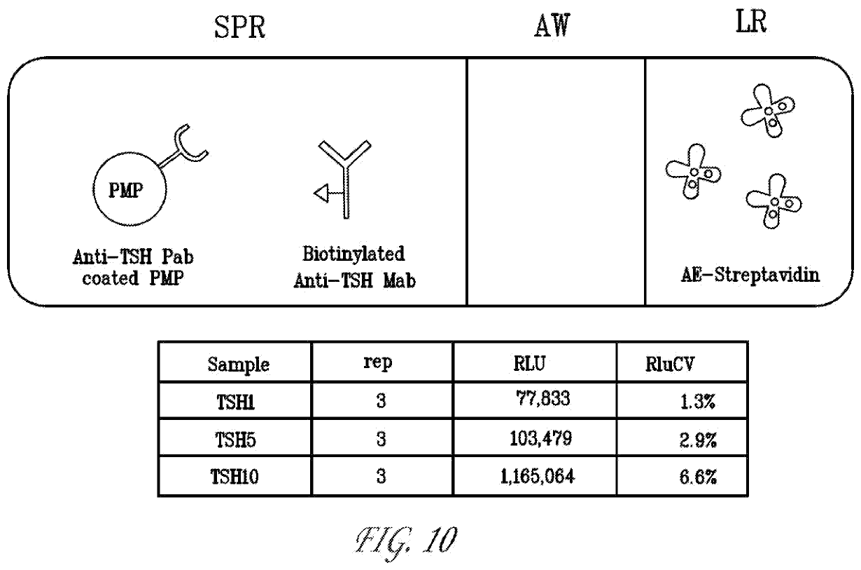

As shown in FIG. 10, the disclosed linklification system can be performed on an automated clinical system such a system tracking thyroid stimulating hormone (TSH). The particles immobilized with anti-TSH Pab (polyclonal antibody) bind the antigen TSH, which then form sandwich with the biotinylated anti-TSH Mab (monoclonal antibody) and the addition of AE-labeled streptavidin produces the signals.

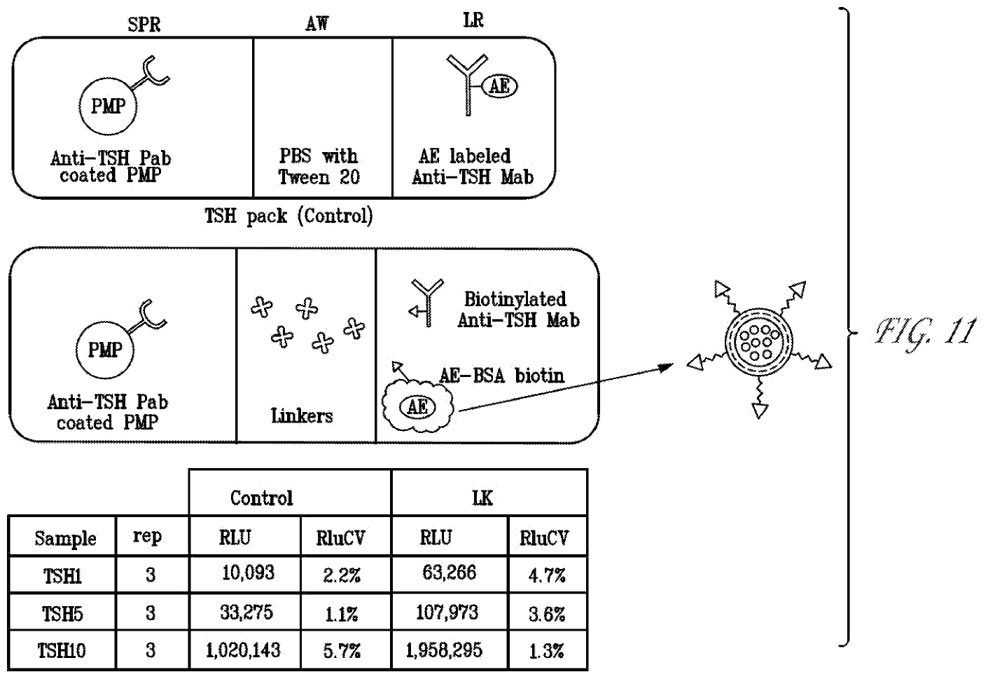

As shown FIG. 11, the linker protein streptavidin, which was unlabeled and placed in a separate reagent compartment (“AW” for ancillary well), allowed linking the biotinylated anti-TSH Mab and the amplifier (AE-BSA-biotin) during the assay. This condition was compared to a control in which AE is directly attached to the anti-TSH Mab.

The disclosed methods and kits can be performed using various immunoassay platforms known in the art, such as but not limited to Siemens' platforms (e.g. ADVIA Centaur® XPT, ADVIA Centaur® XP, ADVIA Centaur® CP, IMMULITE® 1000, IMMULITE 2000 XPi and Atellica®), or General Electric Healthcare platforms (e.g. Biacore®). The presently disclosed methods and kits allow a significant increase in immunoassay signals or relative light units (RLUs) and optimize the assay sensitivity.

The disclosures of each and every patent, patent application, and publication cited herein are hereby incorporated herein by reference in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of detecting an analyte in a sample, the method comprising:
a) combining, in a medium, the sample with a conjugate reagent, a linker reagent, an amplifying reagent, and, optionally, a capture binding partner for the analyte; and
b) examining the medium for bound analyte, the bound analyte comprising the analyte bound to the conjugate reagent bound to the linker reagent bound to the amplifying reagent, wherein the conjugate reagent comprises a detection binding partner for the analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

2. A kit comprising:
a) a conjugate reagent,
b) a linker reagent,
c) an amplifying reagent; and
d) optionally, a capture binding partner, wherein the conjugate reagent comprises a detection binding partner for a target analyte and a first small molecule, wherein the amplifying reagent comprises a labeling agent encapsulated by a liposome or bound to a carrier protein, wherein the liposome or the carrier protein comprises a second small molecule on its surface, and wherein the linker reagent comprises a binding partner for the first small molecule and the second small molecule.

3. The method of claim 1 or the kit of claim 2, wherein the detection binding partner for the analyte comprises an antibody that specifically binds the analyte.

4. The method or kit of claim 3, wherein the first small molecule and the second small molecule comprise biotin.

5. The method or kit according to claim 4, wherein the binding partner for the small molecule comprises streptavidin.

6. The method or kit of claim 3, wherein the first small molecule and the second small molecule comprise fluorescein.

7. The method or kit of claim 6 wherein the binding partner for the small molecule comprises anti-fluorescein antibody.

8. The method or kit of claim 7, wherein the capture binding partner for the analyte further comprises a support.

9. The method or kit of claim 8, wherein the support is a non-magnetic particle, a magnetic particle, a plate, or a tube.

10. The method of claim 9, further comprising a washing step prior to the step of examining the medium for bound analyte.

11. The method or kit of claim 9, wherein the conjugate reagent further comprises a labeling agent.

12. The method or kit of claim 11, wherein the linker reagent comprises a labeling agent.

13. The method or kit of any preceding claim 12, wherein the carrier protein comprises a bovine serum albumin (BSA).

14. The method or kit of claim 13, wherein the labeling agent(s) comprises acridinium ester (AE).

15. The method of claim 13, wherein the sample, the conjugate reagent, the linker reagent, the amplifying reagent, and optionally the capture binding partner are combined simultaneously or sequentially.

16. The method or kit of claim 14, wherein the diameter of the liposome is about 20 nm to about 1000 nm.

17. The method or kit of claim 14, wherein the encapsulated AE has a concentration ranging from at least 1×10⁻⁸ mol/L to at least 1×10⁻⁸ mol/L.

18. The method or kit of claim 14, wherein the liposome encapsulates about 1000 to about 100,000,000,000 hydrophilic AE molecules.

19. The method or kit of claim 14, wherein the carrier protein binds at least 1 to about 100 AE molecules.
